(19) United States
(12) Patent Application Publication
(10) Pub. No.: US 2019/0233788 A1
(43) Pub. Date: Aug. 1, 2019

(54) Production Method For And Use Of Polymer Thin-Film Culture Plat For Production Method For And Application Of Cell Sheet

(71) Applicant: KOREA ADVANCED INSTITUTE OF SCIENCE AND TECHNOLOGY, Daejeon (KR)

(72) Inventors: Sung Gap IM, Daejeon (KR); Eun Jung LEE, Daejeon (KR); Ji Eung BAEK, Daejeon (KR); Young Hak CHO, Daejeon (KR); Seung Jung YU, Daejeon (KR); Go Ro CHOI, Daejeon (KR)

(73) Assignee: Korea Advanced Institute of Science and Technology, Daejeon (KR)

(21) Appl. No.: 16/315,761

(22) PCT Filed: Jul. 5, 2017
(86) PCT No.: PCT/KR2017/007195

§ 371 Date: Jan. 7, 2019

(30) Foreign Application Priority Data
Jul. 5, 2016 (KR) 10-2016-0084856

(51) Int. Cl. C12M 1/22; B05D 7/02; C08J 5/18; C08L 57/06; B05D 1/00

(52) U.S. Cl. CPC C12M 23/10; B05D 7/02; C12N 5/0018; C08L 57/06; B05D 1/60; C08J 5/18

(57) ABSTRACT

The present invention provides a culture plate comprising a copolymer formed from a first monomer for forming a thin film having high surface free energy and a second monomer for forming a thin film having low surface free energy, a method for producing the culture plate, and a method for producing and transferring a cell aggregate in the form of a cell sheet by using the culture plate. The present invention has the effect of producing a cell aggregate in the form of a cell sheet through an easy and simple process in comparison with prior art.

### FIELD

This application claims priority to and the benefit of Korean Patent Application No. 10-2016-0084856 filed in the Korean Intellectual Property Office on 5 Jul. 2016, the disclosure of which is incorporated herein by reference.

The present invention relates to a culture plate and, more specifically, to a culture plate including a polymer thin coating having controllable surface free energy, a method for manufacturing the culture plate, and a method for manufacturing a cell sheet type of cell aggregate by using the culture plate.

### BACKGROUND

In regenerative medicine, tissue engineering therapy has been developed into a therapy by which, on the basis of biodegradable polymers, cells are cultured to rebuild tissues and to be transplanted into damaged or dysfunctional organs for normal functions thereof. However, the transplantation of biodegradable polymer supports cannot avoid problems, such as immune responses and inflammations caused by degradation of biodegradable polymers (Ronneberger B et al., J Biomed Mater Res, 30 (1) (1996), 31-40). There is another technique in which cells are mixed with a biodegradable polymer solution and injected into the human body, but the extracellular matrix (ECM) of cells is broken during the procedure, and as a result, the transplantation of such cells into target tissues results in a marked drop in cell regeneration efficiency (Canavan H et al., J Biomed Mater Res A. 2005 Oct. 1; 75(1): 1-13).

Cell sheets were developed for cell transplantation without supports (Yang J et al., Biomaterials. 2005 November; 26(33):6415-22), and a technique developed by Teruo Okano (Japan) is currently the most used approach, in which poly(N-isopropylacrylamide) (PIPAAm) as a temperature-sensitive polymer is covalently linked to a surface of the polystyrene culture dish to a thickness of 20 nm or less through electron beam irradiation (Tang J et al., Polymers, 2012, 4, 1478-1498, Yang J et al Biomaterials 2007; 28(34):5033-5043, Patent: US2008-0131476-A1). In a temperature-sensitive culture dish, cells adhere thereto to form a cell sheet at a lower critical solution temperature (LCST) or higher, and a sheet type of cells can be collected by polymer swelling at a lower critical solution temperature (LCST) or lower (Haraguchi, Y et al., Nat. Protoc. 7, 850-858 (2012). However, this technique is difficult to universally use and commercialize due to limitations that the temperature of cells needs to be lowered to 20° C. or lower, there are many restrictions in changing chemical functional groups of surfaces, and the fabrication methods are not only difficult but also take a lot of time (Akiyama, Y. et al., Langmuir 20, 5506-5511 (2004).

Studies on use of electric stimulations, ultrasonic stimulations, and pH sensitive in addition to the temperature-sensitive culture dishes have been conducted. However, metal materials (Au, Ag, or CNT) need to be mainly used in order to induce electric stimulation and ultrasound effects, causing the difficulty in coating and expensive equipment in the manufacturing of substrates for cell culture, so that there are restrictions on the use of such metal materials (Guillaume-Gentil O et al., Adv. Mater. 2008, 20, 560-565, Guillaume-Gentil O et al., Biomaterials. 2011, 32, 4376-4384. Hong Y et al., Biomaterials. 2013, 34(1), 11-18 L. Junge et al., Ultrasound in Med. &Biol., 2003, 29, 1769-1776, Sada T et al., ACS Nano, 2011, 5, 4414-4421, Kolesnikova T et al., ACS Nano, 2012, 6, 9585-9595).

Even though cell sheets were fabricated, the sheets need to be repeatedly stacked, and the sheets need to be efficiently transferable to target sites of application thereof in order to clinically apply the sheets in practice. However, the methods known until now have still disadvantages of a lack of reproducibility in stacking and transfer, complicated procedures, and long consuming time. Particularly, most of the existing cell sheet transfer methods adopted stacking through individual transfer of single-layer cell sheets rather than transfer of several cell sheets at a time. There is a need to develop a novel transfer method for cell sheets, capable of stacking cell sheets more easily and quickly as well as transferring the cell sheets to different substrates and disease models.

### REFERENCES

(Patent Document 0001) Korean Patent Registration No. 10-1583159
(Patent Document 0002) Korean Patent Publication No. 10-2016-0056040
(Patent Document 0003) Korean Patent Publication No. 10-2006-0091301
(Non-Patent Document 0001) Yang J et al., Biomaterials. 2005 November; 26(33): 6415-22
(Non-Patent Document 0002) Tang J et al., Polymers, 2012, 4, 1478-1498
(Non-Patent Document 0003) Yang J et al., Biomaterials 2007; 28(34): 50335043
(Non-Patent Document 0004) Haraguchi, Y. et al., Nat. Protoc. 7, 850-858 (2012)

Throughout the specification, many papers and patent documents are used as references, and the citations thereof are represented. The disclosure of the cited papers and patent documents is incorporated in the present specification by reference in its entirety, to describe a level of the technical field to which the present invention pertains and content of the present invention more clearly.

### SUMMARY

### Technical Problem

The present inventors endeavored to solve the above-described problems. As a result, the present inventors confirmed that various types of cell sheets can be formed and separated by controlling the surface free energy that affects the adhesive strength between cells and culture surfaces, and cell sheets can be stacked since the cell sheets are collected while extracellular matrices are contained in the cell sheets. Furthermore, the present inventors confirmed that the coating performed on a culture plate through initiated chemical vapor deposition (iCVD) is a vapor deposition process, so that there is no restriction on substrates and various functional polymers can be coated within a comparatively short time, leading to great advantages in versatility and commercialization, and additionally, cell sheets can be formed by controlling surface free energy through copolymer coating, and then completed the present invention.

Therefore, an aspect of the present invention is to provide a culture plate.

Another aspect of the present invention is to provide a method for manufacturing a cell sheet type of cell aggregate.

Still another aspect of the present invention is to provide a method for surface modification of a culture plate by using initiated chemical vapor deposition.

The objects of the present invention are not limited the aforementioned objects. Therefore, other objects and advantages of the present invention which are not described will be able to be understood and will be more apparently appreciated by the embodiments of the present invention. In addition, it could be easily appreciated that the object and advantages of the present invention can be implemented by the means and combination described in claims.

### Technical Solution

In accordance with an aspect of the present invention, there is provided a culture plate including a copolymer, the copolymer being formed of: a first monomer for allowing a thin film having low surface free energy to be formed; and a second monomer for allowing a thin film having a higher surface free energy than the thin film formed of the first monomer to be formed.

As used herein, the expression “first monomer for allowing a thin film having low surface free energy to be formed” refers to a monomer having a surface free energy of 60 mJ/m² or lower. The expression “second monomer for allowing a thin film having high surface free energy to be formed” refers to a monomer having a surface free energy of 60 mJ/m² or higher. The expression “copolymer formed of a first monomer and a second monomer” refers to a copolymer having a surface free energy of 30-90 mJ/m² formed using the first monomer and the second monomer.

According to the present invention, the expression also means a case in which the surface free energy of the culture plate is implemented to 30-90 mJ/m² through a homopolymer formed of the first monomer or the second monomer.

As used herein, the expression “culture plate containing a copolymer, the copolymer being formed of a first monomer for allowing a thin film having low surface free energy to be formed and a second monomer for allowing a thin film having high surface free energy to be formed” is used not only to mean a portion of a culture plate, which contains a copolymer formed of a first monomer for allowing a thin film having low surface free energy to be formed and a second monomer for allowing a thin film having high surface free energy to be formed (for example, a culture plate of which a surface is coated with the copolymer) but also to mean that a copolymer per se, formed of a first monomer for allowing a thin film having low surface free energy to be formed and a second monomer for allowing a thin film having high surface free energy to be formed, can be used as a culture plate.

Herein, the first monomer is a monomer having weak cell adhesion and low surface free energy. For example, the first monomer may be a monomer selected from the group consisting of aromatic vinyl-based monomers (e.g., divinylbenzene, vinyl benzoate, styrene, etc.), methacrylate-based monomers (e.g., benzyl methacrylate, cyclohexyl methacrylate, butyl methacrylate, Isopropyl methacrylate, ethylene glycol dimethacrylate, and hydroxyethyl methacrylate), fluorine-based monomers (furfuryl methacrylate and perfluorodecyl acrylate), silazanes or cyclosilazanes with a vinyl group (e.g., 2,4,6,8-tetramethyl-2,4,6,8-tetravinylcyclotetrasiloxane, 1,3,5-trivinyl-1,3,5-trimethylcyclotrisiloxane, hexavinyldisiloxane, etc.), monomers having an epoxy functional group (glycidyl methacrylate, etc.), and monomers used as a crosslinking agent (ethylene glycol dimethacrylate, ethylene glycol diacrylate, di(ethylene glycol)divinyl ether, etc.).

Herein, the second monomer is a monomer having strong cell adhesion and high surface free energy. For example, the second monomer may be a monomer selected from the group consisting of vinyl-based amines (2-vinylpyridine, 4-vinylpyridine, 1-vinylimidazole, 1-vinylpyrrolidone, 2-vinylpyridine, 4-aminostyrene, 9-vinylcabazole, etc.), methacrylate-based amines (2-(dimethylamino)ethyl methacrylate, diethylaminoethyl acrylate, dimethylaminoethyl acrylate, diethylaminoethyl acrylate, etc.), monomers having an acidic functional group (maleic anhydride, methacrylic acid, etc.), acrylamide, methacrylamide, monomers having chlorine-based functional groups (4-vinylbenzyl chloride, 2-chloroethyl acrylate, etc.), cyan-based monomers (cyanoethyl acrylate, vinylbenzyl cyanide, etc.), and vinyl-N-methylpyridinium chloride.

In the present invention, the mixing proportions of the first monomer and the second monomer are not limited when the copolymer is formed of the first monomer and the second monomer. The mixing proportions of the first monomer and the second monomer can be controlled within 1-99%.

Therefore, according to an embodiment of the present invention, the first monomer is a monomer for allowing a thin film having low surface free energy to be formed, and the second monomer is a monomer for allowing a thin film having high surface free energy to be formed.

According to a particular embodiment, the first monomer for allowing a thin film having low surface free energy to be formed adopts divinylbenzene (hereinafter, called “DVB”) and the second monomer for allowing a thin film having high surface free energy to be formed adopts 4-vinylpyridine (hereinafter, called “4VP”). The copolymer formed of the first monomer and the second monomer adopts poly(divinylbezene-co-4-vinylpyridine) (hereinafter, called “PD4V”).

According to another embodiment of the present invention, a cell sheet type of cell aggregate can be manufactured by culturing cells on a copolymer formed of the first monomer for allowing a thin film having high surface free energy to be formed and the second monomer for allowing a thin film having low surface free energy to be formed (FIG. 1).

In accordance with another aspect of the present invention, there is provided a method for manufacturing a cell sheet type of cell aggregate, the method including culturing cells on the culture plate of the present invention.

In accordance with still another aspect of the present invention, there is provided a method for surface modification of a culture plate, the method including: degrading an initiator to form free radicals; subjecting a first monomer and a second monomer to a change polymerization reaction by the free radicals to form a copolymer; and depositing the copolymer on a culture plate to form a thin film, wherein the method employs chemical vapor deposition.

Herein, initiated chemical vapor deposition (iCVD) may be used as chemical vapor deposition, and tert-butyl peroxide (TBPO) may be used as an initiator. The initiator is degraded by heat or electricity to form free radicals, which activate the first monomer and the second monomer, and thus a copolymer is formed by a change polymerization reaction of the monomers, and the polymer is deposited to form a thin film, thereby modifying a surface of the culture plate. The thickness of the polymer thin film of the culture plate is not particularly limited, but may be for example, 5 nm to 500 μm. A too thin or thick support layer may affect efficiency in the formation of thin films and stability of thin films for cell culture.

Since iCVD is a low-temperature and low-vacuum process where the temperature of a substrate surface, on which a polymer thin film is deposited, is maintained between 10-45° C., various polymer coatings can be formed on various plastic culture plates (35-pi and 100-pi dishes, and 6-, 12-, 24-, and 96-well plates) without damage. Conventionally employed liquid-phase processes, such as dip coating and spin coating, have problems of substrate damage caused by solvents, non-uniform coating, and the like. Therefore, the forming of coatings on culture plates through iCVD can solve the coating problems that could not be solved by existing coating methods.

According to the present invention, the shape of the culture plate is not limited since the culture plate is sufficient as long as the culture plate provides any space capable of culturing cells therein. For example, the culture plate may have a shape of a dish (culture dish), a Petri dish or plate (e.g., 6-well, 24-well, 48-well, 96-well, 384-well, or 9600-well microtiter plate, microplate, dip well plate, etc.), a flask, a chamber slide, a tube, a cell factory, a roller bottle, a spinner flask, a hollow fiber, a micro carrier, a bead, or the like. In addition, any material having a supporting property may be used as the culture substrate without limitation, and for example, plastic (e.g., polystyrene, polyethylene, polypropylene, etc.), metal, silicon, and glass may be used for the culture substrate. The structure of a culture plate according to an embodiment of the present invention is shown in FIG. 1.

According to an embodiment of the present invention, the first monomer is a monomer for allowing a thin film having high surface free energy to be formed, and the second monomer is a monomer for allowing a thin film having low surface free energy to be formed.

According to a specific embodiment, the first monomer for allowing a thin film having low surface free energy to be formed adopts DVB; the second monomer for allowing a thin film having high surface free energy to be formed adopts 4VP; and the copolymer formed of the first monomer and the second monomer adopts PD4V.

According to another specific embodiment, when a copolymer is deposited by iCVD, cells can be cultured as a cell sheet type or a cell spheroid type of cell aggregate depending on the injection ratio of the first monomer, DVB, and the second monomer, 4VP, as shown in FIG. 2. The higher injection proportion of DVB can attain surface modification of a culture plate suitable to culture cells as a cell spheroid type of cell aggregate, and the increasing injection ratio of 4VP to DVB can attain surface modification of a culture plate suitable to culture cells as a cell sheet type of cell aggregate. Meanwhile, culture plate surfaces with different contact angles and different surface free energy values can be fabricated by controlling the injection ratio of monomers. The adhesive strength between cells and a culture plate surface varies depending on the surface energy of each copolymer coating, so cells are grown as respective forms, and thus a cell sheet type of cell aggregate can be separated from a surface of the culture plate surface without temperature change.

Cell sheets can be formed by culturing various cells on the culture plate of the present invention. The cells to be cultured are not particularly limited in the present invention, and for example, cells that can be isolated or activated from heart, muscle, liver, bone, marrow, thymus, kidney, spleen, lung, brain, testes, ovary, islet, intestine, ear, skin, gallbladder, prostate, bladder, embryo, immune system, and hematopoietic system can be used. Preferably, such cells include various kinds of stem cells, corneal epithelial cells, nerve cells, vascular endothelial cells, cartilage cells, fibroblasts, osteoblasts, myoblasts, kidney cells, hepatic cells, adipocytes, keratinocytes, muscle cells, myocardial cells, or esophageal epithelial cells.

In accordance with an aspect of the present invention, there is provided a method for stacking and transferring a cell sheet type of cell aggregate by using a holed structure.

The method for method for stacking and transferring a cell sheet type of cell aggregate may include:

(a) allowing at least one layer of cell sheet type of cell aggregate to adhere onto a surface of a holed structure; and

(b) disposing the holed structure such that a cell aggregate-adhering surface of the holed structure faces a site in need of application of the cell aggregate, and then peeling only the holed structure.

According to an embodiment of the present invention, the holed structure refers to a membrane type structure on which a cell sheet type of cell aggregate can be placed such that the cell sheet type of cell aggregate can be easily applied to a place or site in need of application thereof. The holed structure may be selected from the group consisting of a nitrocellulose membrane, a nylon membrane, a polyvinylidene fluoride (PVDF) membrane, a polytetrafluoroethylene (PTFE) membrane, a polycarbonate membrane, a mixed cellulose ester (MCE) membrane, a polyamide membrane, and a polyethersulfone (PES) membrane, wherein the holed structure may have one or more holes. However, the holed structure is not limited thereto, and various kinds of membranes that can be used in the art may be used without limitation.

According to an embodiment of the present invention, it is preferable that the holed structure has one or more holes, but the number of holes and the shape of the holes are not limited. The size of the holes is not limited as long as a cell sheet type of cell aggregate does not pass through a membrane to move downwards even while being placed over the holes of the membrane.

According to another embodiment of the present invention, the method may further include dropping a phosphate buffer solution or a cell culture medium on the opposite surface of the cell aggregate-adhering surface of the holed structure when the holed structure are separated from the cell aggregate.

A structural feature of the holed structure used in the above-described method for transfer of a cell aggregate is that holes are formed in the structure, and the holes prevent excessive adhesive strength between a cell sheet and a membrane and enable effective transfer.

First, the holes relatively reduce the contact area between the membrane and the sheet, thereby decreasing absolute adhesive strength therebetween, facilitating transfer.

Second, the cell sheet and the membrane can be separated from each other more easily when a small amount of phosphate buffer solution or a cell culture medium is allowed to flow through the holes at the time of transfer.

### Advantageous Effects

According to the present invention as set forth above, a cell sheet type of cell aggregate can be produced, separated, and collected through an easy and simple process compared with the conventional art by providing: a culture plate including a copolymer formed of a first monomer for allowing a thin film having high surface free energy to be formed and a second monomer for allowing a thin film having low surface free energy to be formed; a manufacturing method for the culture plate; and a method for manufacturing a cell sheet type of cell aggregate by using the culture plate.

### BRIEF DESCRIPTION OF THE DRAWINGS

FIG. 1 shows a structure of a culture plate according to an embodiment of the present invention.

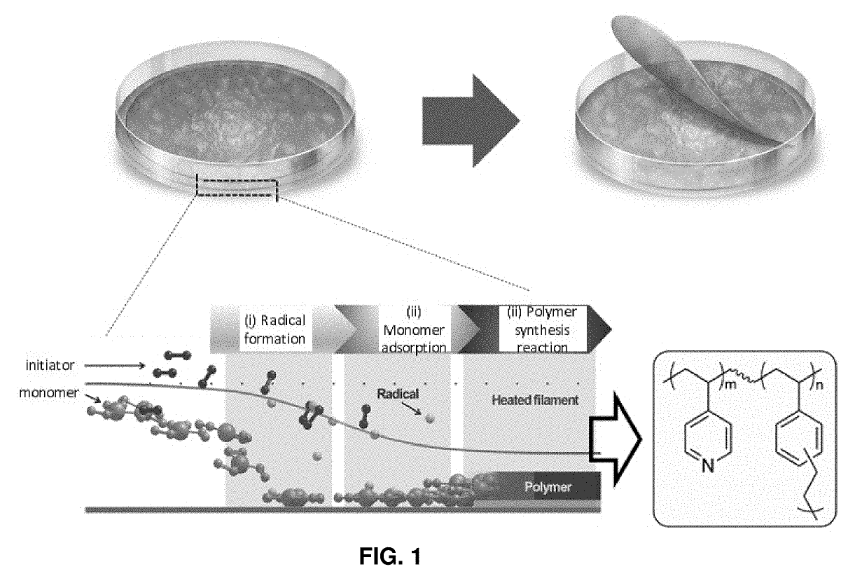

FIG. 2 is a schematic diagram depicting the type of cell aggregate depending on the injection ratio of monomers in the present invention.

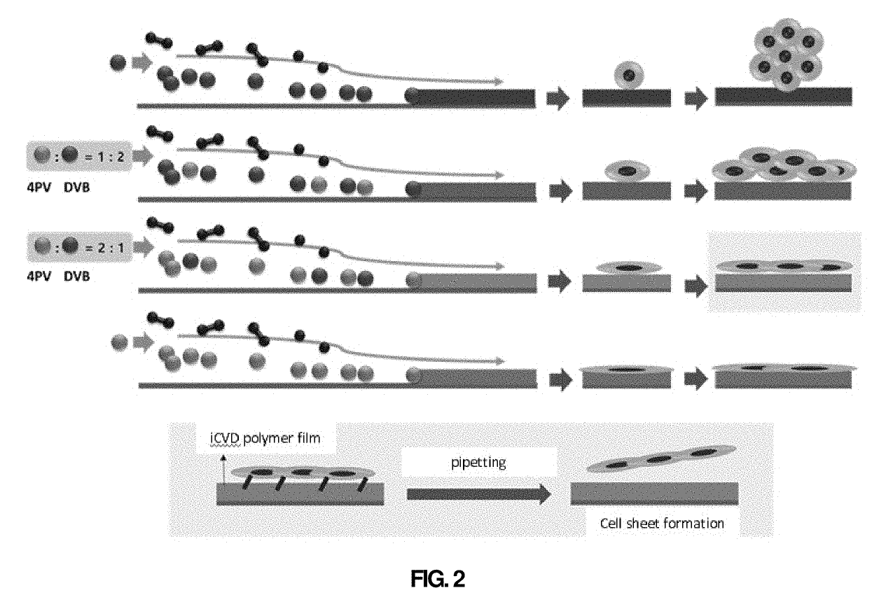

FIG. 3 shows the results of analyzing, using Fourier transform infrared spectroscopy (FT-IR), chemical structures of the surfaces coated with a first monomer polymer (pDVB), a second monomer polymer (p4VP), and pD4V copolymers prepared in an example of the present invention.

FIG. 4 shows the results of measuring the contact angle and surface free energy of the surfaces coated with a first monomer polymer (pDVB), a second monomer polymer (p4VP), and pD4V copolymers prepared in an example of the present invention.

FIG. 5 shows microscopic image results of cells (NIH3T3) cultured on the surfaces coated with a first monomer polymer (pDVB), a second monomer polymer (p4VP), and pD4V copolymers prepared in an example of the present invention.

FIG. 6 shows images depicting that a cell sheet form cultured on a culture plate of the present invention was spontaneously separated from the culture plate by a buffer.

FIG. 7 shows optical and fluorescence microscopic images depicting a procedure in which an adult stem cell sheet (hMSC cell sheet) was formed on the surface coated with pD4V prepared in an example of the present invention, spontaneously separated from the surface, and then collected.

FIG. 8 is a schematic diagram depicting a procedure in which a cell sheet of the present invention was stacked on a holed structure and the resultant structure was applied to a place in need of application of the cell sheet, such as a substrate or a disease model.

FIG. 9 shows images depicting that two cell sheets formed on a culture plate of the present invention were separated, collected, and then stacked.

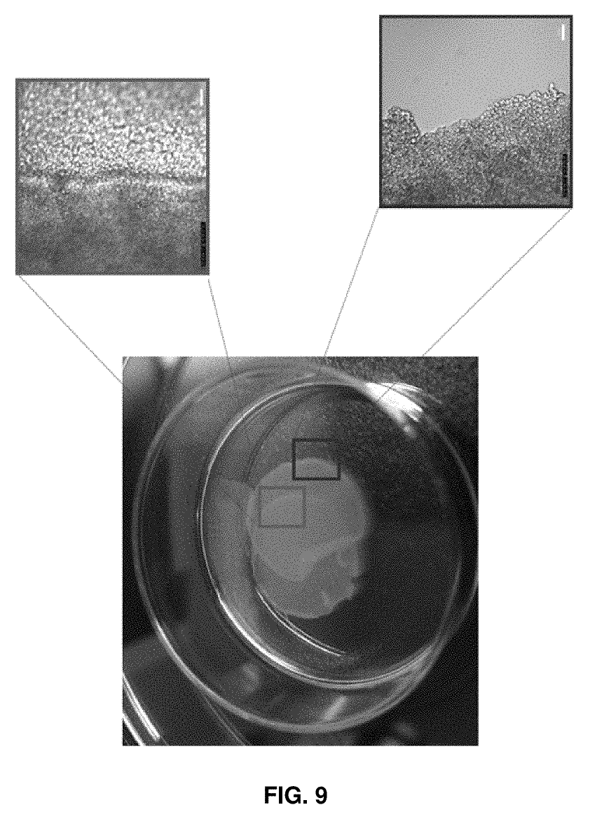

### DETAILED DESCRIPTION

The objects, features, and advantages of the present invention would become apparent through the detailed description that is illustrated later with reference to the accompanying drawings. Therefore, a person having ordinary skill in the technical field to which the present invention pertains would easily implement the technical idea of the present invention. Furthermore, in the description of the present invention, detailed descriptions of known techniques associated with the present invention will be omitted when it is determined that the detailed descriptions unnecessarily obscure the gist of the present invention. Hereinafter, preferred embodiments according to the present invention will be described in detail the with reference to the accompanying drawings.

### EXAMPLE 1

### Preparation of Culture Plate

In the deposition of PD4V, the deposition was carried out for 1 hour and 30 minutes by allowing a divinylbenzene (DVB) monomer (Sigma-Aldrich), a 4-vinylpyridine (4VP) monomer (Sigma-Aldrich), and tert-butyl peroxide (TBPO, Sigma-Aldrich) as an initiator at a ratio of 60:240:60 to flow into an initiated chemical vapor deposition reactor (iCVD, Daeki Hi-Tech Co., Ltd) while a filament temperature of 140° C., a substrate temperature of 23° C., and a chamber pressure of 300 mTorr were maintained in the reactor, and as a result, a culture plate with a 400-nm thick DVB-4VP copolymer (nPD4V) was obtained.

### EXAMPLE 2

### Surface Analysis of Cell Sheet Culture Plate

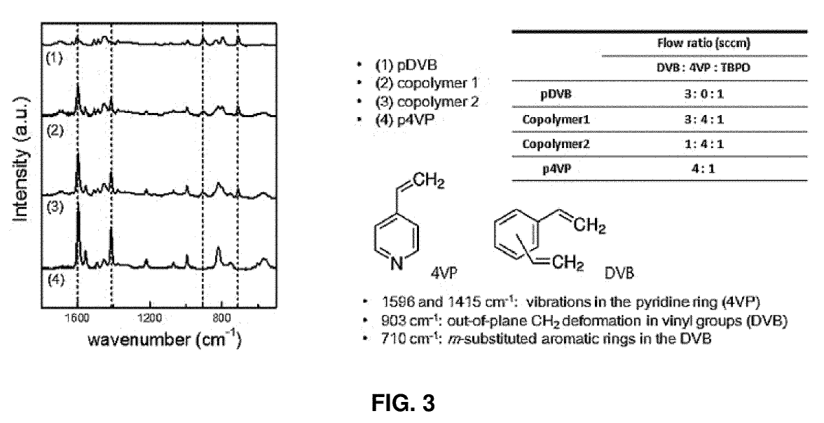

After the deposition of the polymer thin film, the molecular skeleton and fraction of the polymer were measured using Fourier transform infrared spectroscopy (FT-IR, ALPHA FT-IR absorption mode, Bruker Optics). As a result, as shown in FIG. 3, the presence of the 4VP molecule was confirmed through peaks at 1596 cm-1 and 1415 cm-1 (two dot lines on the left side), and the presence of the DVB molecule and the polymer was confirmed through peaks at 710 cm-1 and 903 cm-1 (two dot lines on the right side).

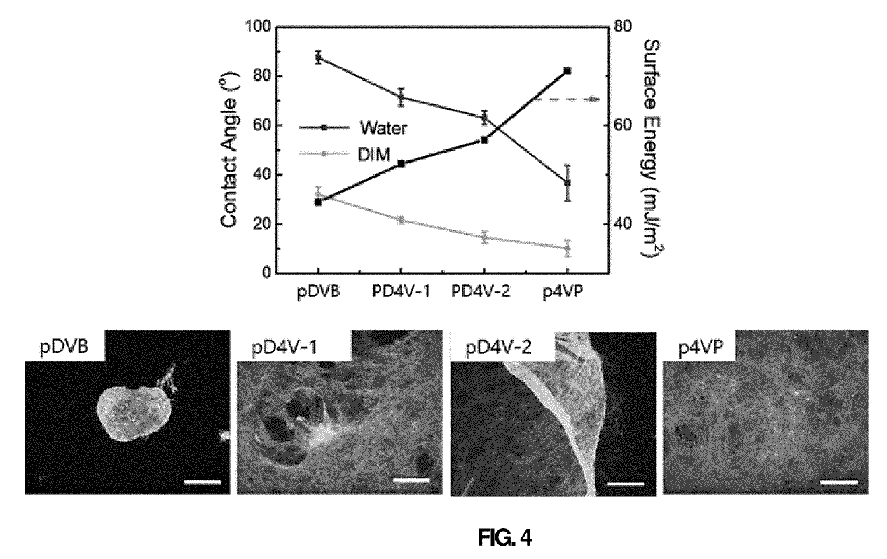

After the deposition of the polymer thin film, the surface contact angles on the substrate relative to 5 μl of distilled water and diiodomethane (DIM) were measured using a contact angle analyzer (Phoenix 150, SEO, Inc.). As a result, it could be verified as shown in FIG. 4 that the surfaces were modified with the polymers formed of different mixing ratio of monomers, leading to different contact angles. On the basis of the results, the surface free energy of the substrate was calculated using Van Oss-Chaudhury-Good (OCG) equation. The results confirmed that the surface free energy value varies depending on the proportion of a polymer.

### EXAMPLE 3

### Observation of Cell Morphology According to Proportion of Copolymer

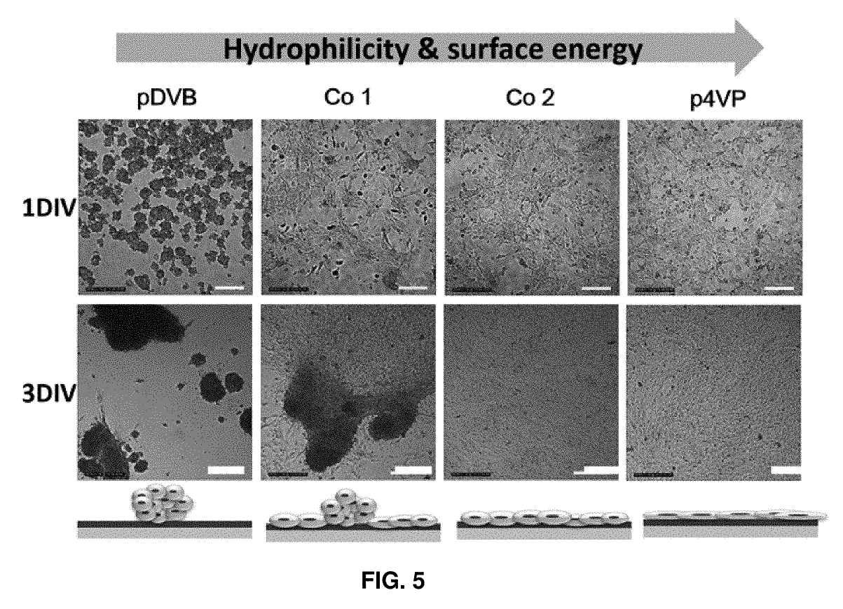

After NIH3T3 cells were cultured on a cell culture dish coated with DVB-4VP copolymer (PD4V), the formation of a cell sheet was investigated. When the cells were sufficiently grown, the cells were fixed with 4% formaldehyde, the nucleus and actin were stained using DAPI and phalloidin, and then the cells were observed by a fluorescence microscope. It was observed as shown in FIG. 5 that the cells grew well without toxicity on all of the culture plates and that spheroids were formed on the DVB culture surface and the cells adhered to and grew on the 4VP culture surface. It was observed that the cells adhered to and grew on the copolymer culture surfaces (pD4V1 and pD4V2), but the cells were spontaneously separated as a cell sheet type after washing with Dulbecco's Phosphate Buffered Saline (DPBS).

### EXAMPLE 4

### Formation and Separation of Cell Sheets

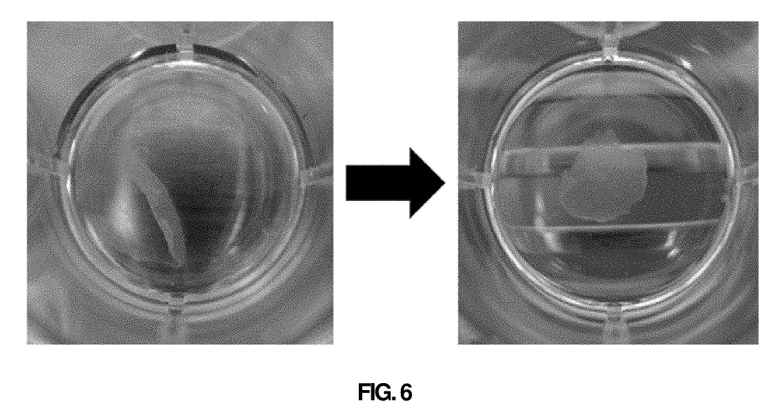

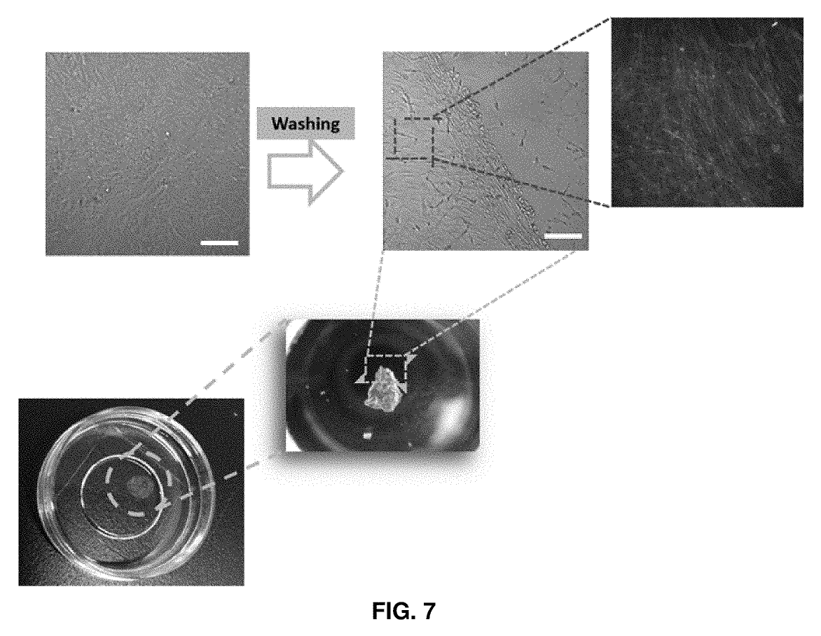

After NIH3T3 and hMSC cells were cultured on 35pi dish on which Pd4V prepared in Example 1 above was deposited, the formation of a cell sheet was investigated. For cell culture, Dulbecco's Modified Eagle Medium (DMEM)/10% FBS/1% antibiotic (penicillin streptomycin, Gibco) were used for NIH3T3 cells, and Minimum Essential Medium α (MEM α)/17% FBS/1% antibiotic (penicillin streptomycin, Gibco) were used for hMSC cells. Cell sheets were formed when the cells were cultured for 3-5 days. Then, the culture liquid was removed, followed by washing with Dulbecco's Phosphate Buffered Saline (DPBS), and here, it could be confirmed that the formed cell sheets were spontaneously separated from the surface of the culture plate (FIGS. 6 and 7).

### EXAMPLE 5

### Separation and Collection of Cell Sheets Formed on Culture Plate and then Stacking of Cell Sheets

In order to manufacture cell sheets, cells were cultured on a cell culture plate coated with PD4V polymer until the intercellular junction could sufficiently occur. Thereafter, the cultured cells were separated as a sheet type by using Dulbecco's Phosphate Buffered Saline (DPBS) solution. After one cell sheet peeled from the culture dish was adsorbed and then transferred to a new cell culture dish, the cell culture dish was left in an incubator containing saturated steam at 37° C. for a proper time (for example, 15-30 minutes). During the time, the cell sheet adhered onto the culture dish. Next, a second cell sheet immediately after peeling was adsorbed together with a culture liquid into a pipette, and dropped on the first cell sheet fixed on the culture dish. A new culture liquid was again slowly dropped on the two dropped sheets, so that the two sheets could join in a state in which the second sheet overlapped the first sheet. Cell sheets could be sequentially stacked by repeating the same procedure.

### EXAMPLE 6

### Use of Holed Structure when Cell Sheets were Stacked and then Transferred

In order to stack the cell sheets manufactured according to the present invention and then more easily transfer the staked sheets to another cell culture dish or a subject in need of application of cell sheets, the following method was used.

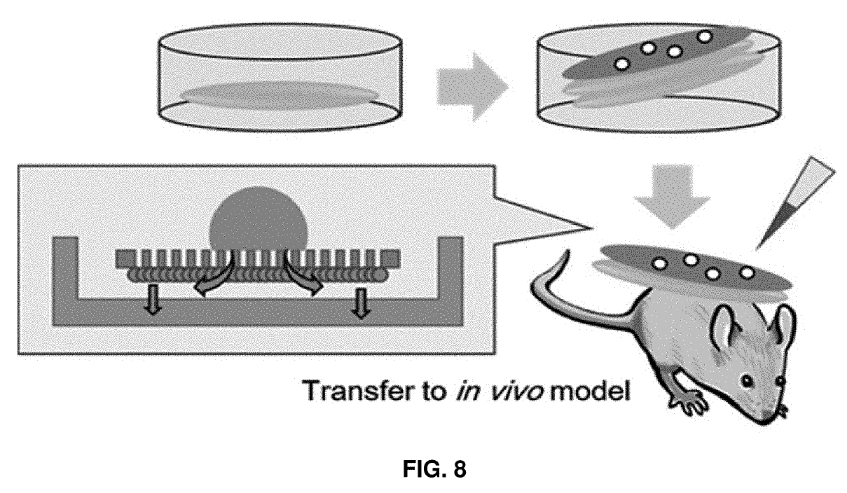

First, as shown in FIG. 8, one cell sheet peeled from the culture dish was dropped to adhere onto a surface of a holed structure (for example, a nitrocellulose membrane with one or more holes). Then, another peeled cell sheet was additionally dropped to adhere to a surface of the cell sheet, which had already adhered to the nitrocellulose membrane, and thus two cell sheets were stacked. A desired number of cell sheets can be stacked by repeating the same procedure.

After the two stacked cell sheets, together with the membrane, were transferred to a new cell culture dish, the cells were incubated in an incubator containing saturated steam at 37° C. for a proper time (for example, 5-30 minutes). As a result, the stacked cell sheets adhered onto the culture dish, and are separated from the membrane as the holed structure. As such, cell sheets, while being stacked on a holed structure, can be transferred to a desired site.

The present invention described above can be made into various substitutions, transformations, and modifications to a person having ordinary knowledge in the technical field to which the present invention pertains without departing from the spirit and scope of the present invention, and therefore, the present invention is not limited to the above-described examples and accompanying drawings.

1. A culture plate comprising a copolymer, the copolymer being formed of:
a first monomer for allowing a thin film having low surface free energy to be formed; and
a second monomer for allowing a thin film having a higher surface free energy than the thin film formed of the first monomer to be formed.

2. The culture plate of claim 1, wherein the first monomer has a surface free energy of 60 mJ/m² or lower.

3. The culture plate of claim 1, wherein the first monomer is selected from the group consisting of divinylbenzene, vinyl benzoate, styrene, benzyl methacrylate, cyclohexyl methacrylate, butyl methacrylate, isopropyl methacrylate, acryl amide, allyl methacrylate, 2-isocyanatoethyl methacrylate, ethylene glycol dimethacrylate, di(ethylene glycol) methyl ester methacrylate, 2-hydroxyethyle methacrylate, 1,2,4-trivinylcyclohexane, furfuryl methacrylate, tetrahydrofurfuryl methacrylate, hexyl methacrylate, hydroxyethyl methacrylate, glycidyl methacrylate, propargyl methacrylate, 1,4-butanediol divinyl ether, isobornyl acrylate, ethylene glycol diacrylate, propargyl acrylate, 2,4,6,8-tetramethyl-2,4,6,8-tetravinylcyclotetrasiloxane, hexavinyldisiloxane, Hexavinyldisiloxane, 1,3,5-trivinyl-1,3,5-trimethylcyclotrisiloxane, 2,4,6-trimethyl-2,4,6-trivinylcyclotrisilazane, dimethylphenylvinylsilane, heptadecafluorodecyl methacrylate, perfluorodecyl acrylate, heptafluorobutyl methacrylate, 1,1,1,3,3,3-Hexafluoroisopropyl methacrylate, 2,2,3,3,4,4-hexafluoro-1,5-pentyl dicrylate, 2-perfluorohexylethyl methacrylate, 2,2,2-trifluoroethyl methacrylate, pentafluorophenyl methacrylate, 1H,1H,7H-dodecafluoroheptyl acrylate, 1H,1H,2H,2H-heptadecaflurodecyl acrylate, di(ethyleneglycol)di(vinyl) ether, 1,9-decadiene, methacrylic anhydride, 1,2,4-trivinylcyclohexane, 2-(methacryloyloxyl)ethyl acetoacetate, allyl acetoacetate, and maleic anhydride.

4. (canceled)

5. The culture plate of claim 1, wherein the second monomer has a surface free energy of 60 mJ/m² or higher.

6. The culture plate of claim 1, wherein the second monomer is selected from the group consisting of 2-vinylpyridine, 4-vinylpyridine, vinylimidazole, vinylpyrrolidone, 4-aminostyrene, 9-vinylcabazole, 2-(diethylamino)ethyl acrylate, diethylaminoethylacrylate, dimethylaminoethylacrylate, diethylaminoethylacrylate, 2-chloroethyl acrylate, cyanoethyl acrylate, 3-(dimethylamino)propyl acrylate, 2-(dimethylamino)ethyl methacrylate, t-butylaminoethyl methacrylate, dimethylaminomethyl styrene, methacrylic acid, acrylamide, methacrylamide, N,N-dimethylacrylamide, N-isopropylacrylamide, 4-vinylbenzyl chloride, vinyl benzyl cyanide, vinyl-N-methylpyridinium chloride, N-vinylcaprolactam, allylamine, N-(4-vinylbenzyl)-N-dimethylamine, and acrylonitrile.

7. (canceled)

8. The culture plate of claim 1, wherein the copolymer has a surface free energy of 30-90 mJ/m².

9. The culture plate of claim 1, wherein the culture plate is for manufacturing a cell sheet type of cell aggregate.

10. The culture plate of claim 1, wherein a material for the culture plate is selected from the group consisting of glass, a metal, a metal oxide, a fiber, paper, and plastic.

11. The culture plate of claim 10, wherein the plastic is selected from the group consisting of polyethylene (PE), polypropylene (PP), polystyrene (PS), polyethylene terephthalate (PET), polyamides (PA), polyester (PES), polyvinyl chloride (PVC), polyurethanes (PU), polycarbonate (PC), polyvinylidene chloride (PVDC), polytetrafluorethylene (PTFE), polyetheretherrketone (PEEK), and polyetherimide (PEI).

12. (canceled)

13. A method for surface modification of a culture plate, the method comprising:
degrading an initiator to form free radicals;
subjecting a first monomer and a second monomer to a change polymerization reaction by the free radicals to form a copolymer; and
depositing the copolymer on a culture plate to form a thin film,
wherein the method employs initiated chemical vapor deposition (iCVD).

14. The method of claim 13, wherein the first monomer has a surface free energy of 60 mJ/m² or lower.

15. The method of claim 13, wherein the first monomer is selected from the group consisting of divinylbenzene, vinyl benzoate, styrene, benzyl methacrylate, cyclohexyl methacrylate, butyl methacrylate, isopropyl methacrylate, acryl amide, allyl methacrylate, 2-isocyanatoethyl methacrylate, ethylene glycol dimethacrylate, di(ethylene glycol) methyl ester methacrylate, 2-hydroxyethyle methacrylate, 1,2,4-trivinylcyclohexane, furfuryl methacrylate, tetrahydrofurfuryl methacrylate, hexyl methacrylate, hydroxyethyl methacrylate, glycidyl methacrylate, propargyl methacrylate, 1,4-butanediol divinyl ether, isobornyl acrylate, ethylene glycol diacrylate, propargyl acrylate, 2,4,6,8-tetramethyl-2,4,6,8-tetravinylcyclotetrasiloxane, hexavinyldisiloxane, Hexavinyldisiloxane, 1,3,5-trivinyl-1,3,5-trimethylcyclotrisiloxane, 2,4,6-trimethyl-2,4,6-trivinylcyclotrisilazane, dimethylphenylvinylsilane, heptadecafluorodecyl methacrylate, perfluorodecyl acrylate, heptafluorobutyl methacrylate, 1,1,1,3,3,3-Hexafluoroisopropyl methacrylate, 2,2,3,3,4,4-hexafluoro-1,5-pentyl dicrylate, 2-perfluorohexylethyl methacrylate, 2,2,2-trifluoroethyl methacrylate, pentafluorophenyl methacrylate, 1H,1H,7H-dodecafluoroheptyl acrylate, 1H,1H,2H,2H-heptadecaflurodecyl acrylate, di(ethyleneglycol)di(vinyl) ether, 1,9-decadiene, methacrylic anhydride, 1,2,4-trivinylcyclohexane, 2-(methacryloyloxyl)ethyl acetoacetate, allyl acetoacetate, and maleic anhydride.

16. (canceled)

17. The method of claim 13, wherein the second monomer has a surface free energy of 60 mJ/m² or higher.

18. The method of claim 13, wherein the second monomer is selected from the group consisting of 2-vinylpyridine, 4-vinylpyridine, vinylimidazole, vinylpyrrolidone, 4-aminostyrene, 9-vinylcabazole, 2-(diethylamino)ethyl acrylate, diethylaminoethylacrylate, dimethylaminoethylacrylate, diethylaminoethylacrylate, 2-chloroethyl acrylate, cyanoethyl acrylate, 3-(dimethylamino)propyl acrylate, 2-(dimethylamino)ethyl methacrylate, t-butylaminoethyl methacrylate, dimethylaminomethyl styrene, methacrylic acid, acrylamide, methacrylamide, N,N-dimethylacrylamide, N-isopropylacrylamide, 4-vinylbenzyl chloride, vinyl benzyl cyanide, vinyl-N-methylpyridinium chloride, N-vinylcaprolactam, allylamine, N-(4-vinylbenzyl)-N-dimethylamine, and acrylonitrile.

19. (canceled)

20. The method of claim 13, wherein the copolymer has a surface free energy of 30-90 mJ/m².

21. The method of claim 13, wherein the culture plate is for manufacturing a cell sheet type of cell aggregate.

22. A method for transfer of a cell aggregate, the method comprising:
(a) allowing at least one layer of cell sheet type of cell aggregate to adhere onto a surface of a holed structure; and
(b) disposing the holed structure such that a cell aggregate-adhering surface of the holed structure faces a site in need of application of the cell aggregate, and then peeling only the holed structure.

23. The method of claim 22, wherein the holed structure is selected from the group consisting of a nitrocellulose membrane, a nylon membrane, a polyvinylidene fluoride (PVDF) membrane, a polytetrafluoroethylene (PTFE) membrane, a polycarbonate membrane, a mixed cellulose ester (MCE) membrane, a polyamide membrane, and a polyethersulfone (PES) membrane, and
wherein the holed structure has one or more holes.

24. The method of claim 22, wherein in the peeling of the holed structure in step (b), a phosphate buffer solution or a cell culture medium is dropped on the opposite surface of the cell aggregate-adhering surface of the holed structure.
